(19) United States
(12) Patent Application Publication
(10) Pub. No.: US 2008/0208236 A1
(43) Pub. Date: Aug. 28, 2008

(54) DERMAL MARKING FOR USE WITH A MEDICAL DEVICE

(75) Inventors: Eamonn P. Hobbs, Queensbury, NY (US); William M. Appling, Granville, NY (US)

Correspondence Address: ANGIODYNAMICS, INC., 603 QUEENSBURY AVENUE, QUEENSBURY, NY, 12804, US

(73) Assignee: AngioDynamics, Inc., Queensbury, NY (US)

(21) Appl. No.: 12/039,043

(22) Filed: Feb. 28, 2008

Related U.S. Application Data
(60) Provisional application No. 60/892,057, filed on Feb. 28, 2007.

(51) Int. Cl. A61M 37/00

(52) U.S. Cl. 606/186; 128/898

(57) ABSTRACT

A dermal marking and a method of identifying the characteristics of an implanted medical device using the dermal marking. The method involves providing an implantable medical device, implanting a medical device in a patient, providing at least one dermal marking that contains information disclosing at least one characteristic of the implanted medical device, applying the at least one dermal marking to the skin of the patient, perceiving the dermal marking on the patient's skin, and identifying at least one characteristic of the implantable medical device based on the dermal marking.

### CROSS-REFERENCE TO RELATED APPLICATIONS

This application claims priority to and the benefit of U.S. Provisional Application No. 60/892,057, filed on Feb. 28, 2007, which application is incorporated in its entirety in this document by reference.

### BACKGROUND OF THE INVENTION

### Field of the Invention

The present invention relates to the field of medical devices. Particularly, the present invention pertains to methods for identifying the characteristics of implantable medical devices. More particularly, the present invention pertains to methods for identifying implanted medical devices, such as vascular access ports or peripherally inserted central catheters (PICCs), in a patient body.

Medical devices such as implanted vascular access devices provide venous access to the central circulatory system of a patient. Vascular access ports are vascular access devices that are implanted into a patient's vascular system for applications in which repeated access to a patient's vascular system is necessary. Repeated access could be for the purpose of infusion of medications, prolonged intravenous feeding of fluids such as drugs or other fluids, parenteral nutrition solutions, blood products, imaging solutions, chemotherapy treatments, intensive antibiotic treatment, withdrawal of blood samples, or extracorporeal blood treatment protocols. Vascular access ports were developed to overcome problems associated with limited peripheral access in patients and to address the need for frequent venipuncture in patients with long-term venous therapies.

Typically, a vascular access port has a reservoir, an inlet septum in the center of the reservoir, and is fluidly connected to an outlet catheter that is placed into a vein in a patient. To implant the vascular access port in a patient, the distal tip of the catheter is introduced through an incision site in a patient, and the port to which the catheter is connected is placed underneath the patient's skin, preferably in an area that is supported by firm underlying tissue, provides good stability, and does not interfere with patient mobility or create any additional pressure. Typically, the catheter is placed into the superior vena cava through the subclavian, jugular, or cephalic vein.

As one skilled in the art will appreciate, the port septum is generally positioned at a depth relative to the skin membrane that aids in ensuring the ease of locating and inserting a needle into the port septum and in deterring tissue erosion due to pressure necrosis. In one exemplary aspect, the tissue thickness over the port septum can be between approximately 0.5 cm to approximately 2 cm. The port septum may then be percutaneously accessed through the patient's skin by using a needle, such as, for example, and not meant to be limiting, a non-coring needle, sometimes referred to as a Huber-style needle. The needle is penetrated through the patient's skin that overlays the access port and is inserted further through the port septum and into the reservoir. The needle is used to deliver fluids, such as, for example, and not meant to be limiting, medications and drugs, into the port. In operation, the fluids exit through the reservoir and into the catheter, allowing treatment to be delivered directly into the bloodstream. In another aspect, blood samples can also be taken in this way if needed.

Other types of implantable medical devices include peripherally inserted central catheters (PICCs), which typically provide long term peripheral access to the central venous system for intravenous therapy, blood sampling and CT (computer tomography) injections. A PICC is inserted into a peripheral vein, such as the cephalic vein, basilic vein, or brachial vein and then advanced through increasingly larger veins, toward the heart until the tip rests in the distal superior vena cava or cavo-atrial junction.

Vascular access ports and PICCs are commonly used as vehicles for performing diagnostic imaging of blood vessels during CT imaging. This procedure involves injecting into a blood vessel a dye, or contrast media, that is visible under fluoroscopic or CT imaging. CT imaging takes a rapid stream of X-ray photographs from different angles. Through computerization, this block of data is used to create two- and three-dimensional images of bone and other hard tissue, and soft tissue when contrast media is introduced inside the body.

CT injectors are used in CT imaging to inject a controlled volume of contrast media or dye at a high pressure into catheters, such as PICCs, that are inserted into a blood vessel. When the dye is injected it mixes with the blood, makes the blood less permeable by x-ray, and allows the vasculature to be visualized. Some advantages of intravenously delivering contrast media by means of power injection for CT include uniform contrast-medium delivery, optimal timing of delivery, and decreased radiation and needle exposure to health-care providers.

Because implanted medical devices may require frequent access during patient treatment, it is important for a medical practitioner to be able to identify the type and characteristics of the device. Such characteristics could include, for example, whether the device is or is not CT injectable, or any other special features of the device, to name a few. This information may be critical because, for example, if a medical practitioner mistakenly uses a non-CT injectable port for CT injections, serious complications could occur. For instance, the implanted medical device could rupture or burst, causing extravasation of contrast dye or chemotherapeutic agents or leaking. This could require that the entire medical device may have to be removed and replaced with a new medical device. This could also result in injury to the patient, or at the very least, compromise patient treatment.

Thus, once a medical device is implanted in the patient's skin, it is critical that the physician or medical practitioner be able to quickly and correctly identify the characteristics of a medical device on a regular basis, with a minimal amount of time and effort. Therefore, it is advantageous to have a means of quickly and reliably identifying the characteristics of an implanted medical device in order to avoid having to rely upon trial and error, lengthy procedure times, or potential trauma to the patient.

To accomplish this, several different methods of identifying medical devices have been proposed. However, many of these methods can be time-consuming because they require multiple steps, additional equipment, or they can be difficult to accomplish in a wet or slippery environment while wearing gloves. Different tactile, visual, or electronic methods have been proposed to assist a practitioner in identifying implantable medical devices. Such exemplary proposed methods could include tactile palpation of physical indicators on the medical device itself, visually perceiving patient chart stickers that contain information pertaining to the medical device, the use of radiopaque or other CT visible marker elements, and/or the use of electronic means to locate or identify the medical device. Although these methods are all viable, they have several disadvantages.

Radiopaque markers or other CT visible marker elements, labels, or other indicia may be engraved on or attached to an implantable medical device to indicate the type or characteristics of the device, in addition to the position of the device. These indicia may provide a high-contrast image when visualized under fluoroscopy, ultrasound, or some other surgical imaging modality. These identification mechanisms can increase the cost of the implantable medical device, and may adversely increase procedure costs and time. The use of such incorporated indicia to identify the port type or other characteristic also leaves room for error by the medical practitioner because the practitioner must be able to translate the marker or the information conveyed by the marker to a characteristic of the implantable device.

Palpation of implanted medical devices involves attempting to manually identify the medical device through the surface of the skin. Typically, physical features are added to the exterior of the medical device, such as protrusions or ridges, to aid in identification of the medical device. However, palpation of these features may not be effective identifiers of the medical device because they may be difficult to locate. For instance, the bumps or protrusions may be too small or implanted too deeply within the skin, for example, in the case of large or obese patients for the “features” to be readily felt. Additionally, even if a medical practitioner is able to locate the bumps or protrusions, the protrusion patterns must be repeatedly associated with a specific feature of the device each time the device is palpated.

Existing electronic methods of identifying an implanted medical device are typically cost prohibitive and may lengthen the procedure time. Furthermore, electronic methodologies could be unreliable because they generally have components with finite lifespans, such as batteries which could die or circuits which could short out. These problems could make it difficult for the practitioner to consistently identify an implanted medical device, and could also adversely require that the medical device be removed and/or re-implanted.

Alternatively, body stickers have been proposed to locate reference positions on a patient's skin for the purpose of surgical treatment. Other identifiers, such as key chains, wrist bands, bracelets, patient ID cards, patient chart stickers, or other temporary labels have been proposed to be used to identify implanted medical devices. However, these identifiers are disadvantageous because they may fall off the patient's skin or become lost, thereby decreasing the effectiveness of such means.

Felt tip markers have been regularly used to indicate places of incision during surgery. However, marks made on the skin using markers may become smudged or inadvertently removed during treatment, such as by dissolving naturally or from exposure to bodily fluids such as the patient's perspiration or natural oils, or site care solutions, such as antiseptics. It can also be difficult to provide uniform and legible handwritten markings when using a marker. For example, a handwritten mark by one medical practitioner may be misinterpreted or confused by another medical practitioner, which could lead to mistakes during treatment, thereby compromising patient safety. Other types of labels, such as tattoos, have been used in medical procedures, such as radiation therapy for cancer, re-pigmentation of the skin, or breast reconstruction. However, all of the above-mentioned methods of marking have been generally used to indicate location or reference points, and not to identify the type or characteristics of an implanted medical device.

Current medical device identification methods are not always sufficiently reliable. Nor do they always allow a minimal amount of effort on the part of the practitioner. Thus, there has been, and continues to be, a need for a solution to the above mentioned problems, such as a reliable method of identifying the characteristics of an implanted medical device that requires minimal tactile or visual effort and eliminates the need to interpret the meaning of markers or other indicia that are attached to or part of an implantable medical device. A solution to the above-mentioned problems would be a method of identifying the characteristics of an implanted medical device from the surface of a patient's skin using a reliable biocompatible semi-permanent or permanent dermal marking.

The present invention addresses the problems in the prior art by providing a method of reliably identifying the type and characteristics of a subcutaneously implanted medical device, using a dermal marking, such as a tattoo, that is imprinted on the patient's skin through the epidermis and/or or dermis of a patient's skin. This would avoid the problems and difficulties associated with the currently proposed methods described above. Such a marking could be comprised of words, phrases, letters, pictures, symbols, or other markings that contain information pertaining to certain characteristics of the implanted medical device. This would provide a cost-effective, easy, and reliable method of identifying the characteristics of an implanted medical device.

Accordingly, it is a purpose of the present invention to provide a method of identifying the characteristics of an implanted medical device using a dermal marking that is located on the surface of a patient's skin.

Various other objectives and advantages of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description of the Invention.

### BRIEF SUMMARY OF THE INVENTION

The present invention provides a dermal marking and a method of identifying the characteristics of an implanted medical device using the dermal marking, such as a tattoo. In one exemplary aspect, a dermal marking is provided for use with an implantable medical device. The dermal marking has information about the characteristics of the implantable medical device, such as CT injection capability.

In another exemplary aspect, the method of the present invention involves one or more of the following steps: providing at least one implantable medical device, implanting the medical device in a patient, providing at least one dermal marking that has information disclosing at least one characteristic of the medical device, applying the dermal marking to the patient's skin, perceiving the dermal marking on the patient's skin, and identifying the at least one characteristic of the implanted medical device based on the dermal marking.

The present invention also provides a method of identifying the characteristics of an implanted medical device using a scanning device. The method involves one or more of the following steps: providing at least one dermal marking that has scannable information disclosing at least one characteristic of the medical device, scanning the dermal marking on the patient's skin using a scanning device, perceiving output data produced from the scanning device pertaining to the dermal marking, thereby identifying at least one characteristic of the implanted medical device based on the dermal marking.

In a further aspect, the present invention can also provide a kit that can include, but is not limited to, an implantable medical device, at least one dermal marking, along with instructions for use, in a container and enclosed in a sealable packaging.

In various aspects, the method of the present invention is advantageous in view of current methods of identifying the characteristics of an implanted medical device because it provides an easier, more cost-effective method of permanently or semi-permanently marking the patient's skin with an indicator that conveys information about the type or characteristics of the implanted medical device. The method of the present invention allows a medical practitioner to quickly, reliably, and correctly identify the characteristics of an implanted medical device with less trial and error, thereby reducing procedure time and potential complications. It is contemplated that the method of the present invention provides a viable alternative to the use of markers, stickers, or other less reliable methods of identifying the characteristics of an implanted medical device.

### BRIEF DESCRIPTION OF THE SEVERAL VIEWS OF THE DRAWINGS

The foregoing advantages and features, as well as other advantages and features, will become apparent with reference to the description and accompanying figures below, which are included to provide an understanding of the invention and constitute a part of the specification, in which like numerals represent like elements, and in which:

FIG. 1 is a partial exploded perspective view of an implanted medical device, such as a vascular access port, positioned beneath a patient's skin, showing one embodiment of a dermal marking located on the patient's skin in the general vicinity of, and surrounding at least a portion of the implanted medical device, in accordance with the present invention.

FIG. 2 is a partial exploded perspective view of an implanted medical device, such as a vascular access port, positioned beneath a patient's skin, showing another embodiment of a dermal marking located on the patient's skin in the general vicinity of, and surrounding at least a portion of the implanted medical device, in accordance with the present invention.

FIG. 3 is a partial exploded perspective view of an implanted medical device, such as a vascular access port, positioned beneath a patient's skin, showing yet another embodiment of a dermal marking located on the patient's skin in the general vicinity of, and overlying at least a portion of the implanted medical device, in accordance with the present invention.

FIGS. 4A and 4B are enlarged top plan views of several different embodiments of the dermal marking of the present invention, in accordance with the present invention.

FIGS. 5A and 5B are enlarged top plan views of several different embodiments of the dermal marking, in accordance with the present invention.

FIGS. 6A through 6E are top plan views of several different embodiments of the dermal marking of the present invention, in accordance with the present invention.

### DETAILED DESCRIPTION OF THE INVENTION

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention.

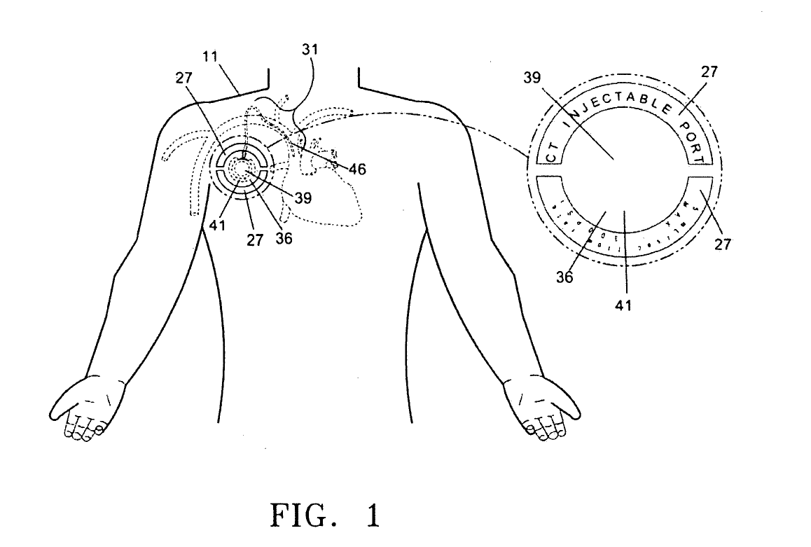

Referring to FIG. 1, an implanted vascular access port assembly 31 is shown implanted beneath the patient's skin 11. Although a vascular access port 36 is illustrated as an example of an implantable medical device in FIG. 1, it is contemplated that any suitable insertable or implantable medical device may be used in the present invention, such as, but not limited to, PICCs, drug infusion pumps, breast implant devices, and the like. Thus, the terms vascular access port 36 and implantable medical device are used interchangeably herein. In various exemplary aspects, the vascular access port 36 of the present invention may be a single or dual vascular access port. It will be appreciated that the dermal marking 27 of the present invention may be used in conjunction with an implantable medical device of any suitable size. Further, any number of implantable medical devices 36 may be used with any number of dermal markings 27.

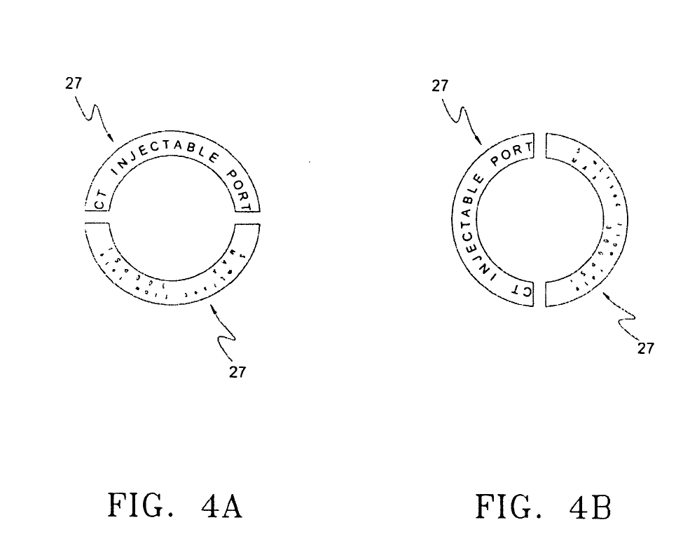

The vascular access port assembly 31 has at least a port housing 41, a septum 39, a reservoir (not shown), and a catheter 46 that is fluidly connected to the septum 39 of the vascular access port 36. The implanted vascular access port 36 can be conventionally placed underneath the skin 11 of the patient using methods well known in the art. In one aspect, at least one dermal marking 27 of the present invention can be applied to the surface of the patient's skin 11 at, surrounding, at least partially surrounding, or in the general vicinity of the implanted vascular access port 36. Alternatively, the dermal marking may be applied anywhere on the patient's body that is reasonably visible by a medical practitioner or other personnel. In one example, the dermal marking 27 may be applied directly above the implanted vascular access port septum 39. In one embodiment, as illustrated in FIG. 1, the dermal marking 27 comprises two separate semi-circle shaped portions that are placed in a substantially surrounding relationship to the implanted port. Optionally, and as illustrated in FIGS. 4A and 4B, the dermal marking 27 may be placed at any desired orientation on the patient's skin relative to the implanted medical device 36. Preferably, the dermal marking 27 is applied to the patient's skin 11 at or in the general vicinity of the implantable medical device 36.

As used herein, the term “dermal marking” 27 means any semi-permanent or permanent pigmented marking that is applied to either the epidermis or the dermis, or both, of a patient's skin 11. Such dermal markings 27 may include, but are not limited to, labels, decals, or tattoos. Preferably, the dermal marking 27 of the present invention is in the form of a tattoo.

It is contemplated that the dermal marking 27 of the present invention may comprise indicia such as, for example, and not meant to be limiting, words, phrases, letters, pictures, symbols (including international medical symbols), or other markings that convey information pertaining to at least one characteristic of the implanted vascular access port 36. Such indicia could comprise information pertaining to the implantable medical device, for example, but not limited to, the type, material, shape, brand, manufacturer, model number, size, mechanical strength, any or all physical or chemical properties of the medical device, capabilities of the device, such as performance and pressure capabilities, inertness, whether the device is a single or dual port, or the maximum flow rate. As one skilled in the art will appreciate, any such provided information may be useful in determining whether the implanted port is useful for CT injections.

Other optional characteristics of the medical device may comprise, but are not limited to, appropriate flush solutions for the device, cleaning or site care solutions, and the volume of the device. Phrases such as, but not limited to, “CT Injectable Port, Max: 300 psi, 5 mL/sec flow rate”, “WARNING: NOT CT INJECTABLE”, as illustrated in FIGS. 4A, 4B, 5B, and 6A through 6E, may be used to indicate the at least one characteristic of the implantable medical device 36. Optionally, the dermal marking 27 may also include patient emergency contact information. In a further aspect, the dermal marking 27 of the present invention may include the shape or design of the dermal marking 27, in combination with the indicia. Alternatively, the dermal marking 27 may simply be the indicia.

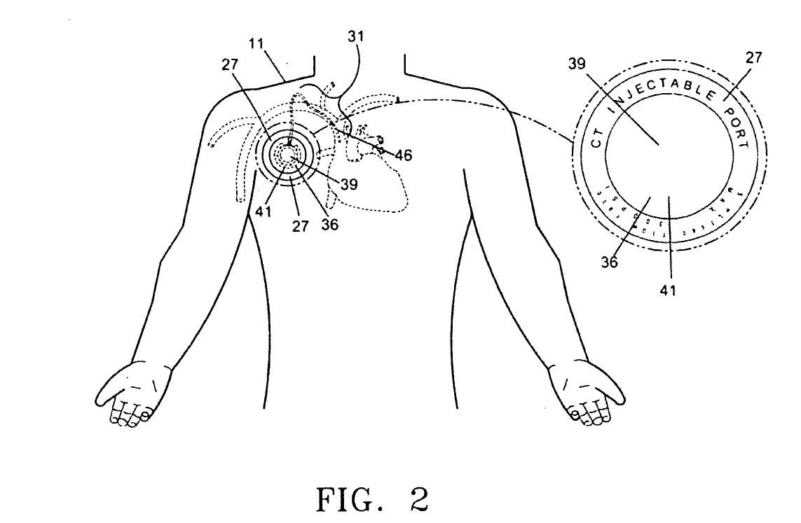

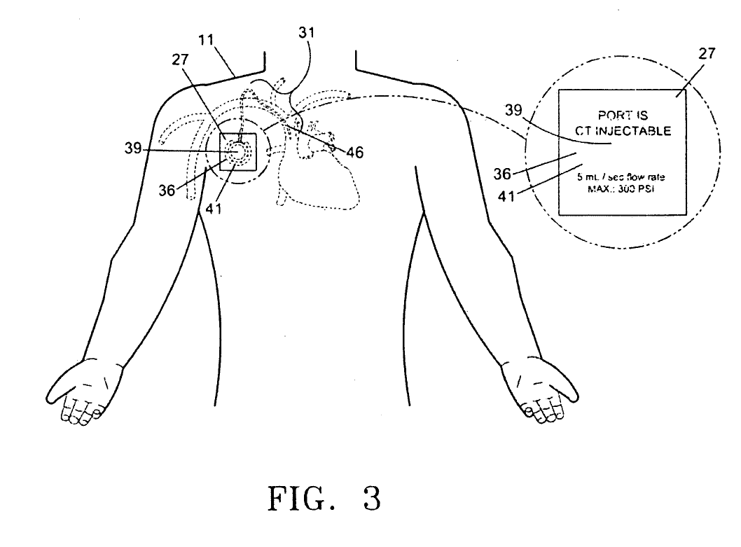

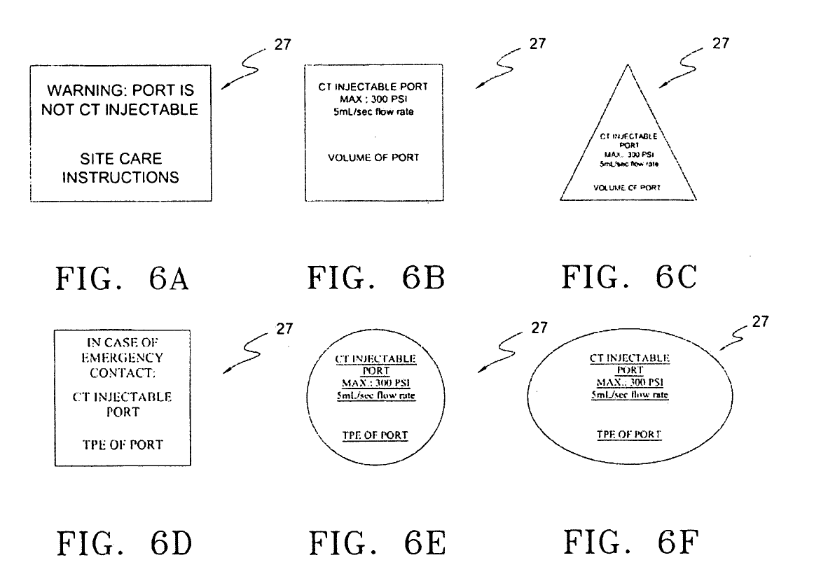

The dermal markings 27 illustrated in FIGS. 1 through 6E are included herein for illustration purposes only and are not intended to be limiting. For example, and referring to FIGS. 6A through 6E, the dermal marking 27 of the present invention may be composed of any suitable color, shape, size, design, pattern, marking, font, or logo, or any suitable combination thereof. Optionally, and as exemplarily illustrated in FIGS. 1, 4A, and 4B, the dermal marking 27 may be composed of multiple portions. In another aspect, the dermal marking 27 may also be composed of a single portion, such as the dermal markings 27 illustrated in FIGS. 2, 3, 5A, 5B, and FIGS. 6A through 6E.

In a further aspect, the dermal marking 27 can be composed of any aesthetically pleasing design. Optionally, the dermal marking 27 design may be varied according to the patient's choice. One of ordinary skill would appreciate that any infinite number of designs may be used in the dermal marking 27 of the present invention.

In various aspects, the font of the dermal marking 27 may be, but is not limited to, Arial, Courier, Times Roman, all capital letters, bold face, italics, underlined text, and the like. It is also contemplated that the words, phrases, letters, symbols, or markings contained in the dermal marking 27 may be in any suitable and recognizable language, such as, but not limited to, Arabic, Chinese, English, French, German, Russian, or Spanish, or any combination of such languages. In one preferred aspect, the dermal marking 27 is in the English language.

The colors used in the dermal marking 27 may be composed of any inks, pigments, or dyes, or any combination thereof, that have been determined to be medically acceptable or biocompatible. In one exemplary aspect, the inks can be composed of hypoallergenic materials in order to minimize the risk of infection, and the dermal marking 27 is MRI safe and compatible. In one aspect, such dyes may be of a permanent nature. Optionally, the dyes used in the dermal marking 27 of the present invention may be made of biodegradable ink, designed for semi-permanent or short term use on the patient's skin.

In one aspect, the dermal marking 27 should be sufficiently capable of being perceived or visualized and should be compatible with a patient's skin type and color. The color of the dermal marking 27 should be clearly visible and different from normal marks found on the patient's skin, so as not to blend in with the patient's skin. The colors used in the dermal marking 27 could include, but are not limited to, gentian violet, Chromium oxide green, 1 Ultramarine blue, acridine, quinoline, phthalocyanine and naphthol derivatives, Logwood extract, dyes made from ash, Ferric ammonium citrate, Phthalocyanine green, Chromium-cobalt-aluminum oxide, brilliant green, methylene blue, vegetable dyes, inorganic materials like titanium dioxide and iron oxide, FD&C Red, FD&C Blue, carbon black, azo dyes, FD&C Green, FD&C Yellow, D&C Orange, D&C Brown, D&C Violet, and any combination of such colors. Alternatively, for patients who desire an invisible tattoo, the dermal marking 27 may be invisible to the naked eye, but visible only under certain wavelengths, such as infrared wavelengths or UVA light, also known as black light.

The dermal marking 27 of FIG. 1 can be used in the method of the present invention to visually identify at least one characteristic of an implantable medical device, such as an implanted vascular access port 36. The method comprises providing an implantable medical device, implanting the medical device beneath a patient's skin 11, and providing at least one dermal marking 27. In this aspect, the at least one dermal marking 27 contains information disclosing at least one characteristic of the implanted medical device. The method can further comprise applying the at least one dermal marking 27 to the patient's skin 11, and perceiving the dermal marking 27 on the patient's skin 11. In this aspect, the dermal marking 27 identifies the at least one characteristic of the implantable medical device. The dermal marking may be placed on or in the skin at or near the vicinity of the implanted medical device, or alternatively, the implanted medical device may be placed on or in the skin anywhere on the patient's body.

Alternatively, the method of the present invention may comprise providing an implantable medical device and providing at least one dermal marking 27 that contains information disclosing at least one characteristic of the implanted medical device. In use, the at least one dermal marking 27 can be selectively applied to the skin 11 of the patient, and the medical device can then be implanted beneath the patient's skin 11. The dermal marking 27 can then be perceived by an external view which allows for the identification of the at least one characteristic of the implanted medical device. The method may further involve identifying the type of device or other features of the device, such as CT injection capability, based on the information perceived from the dermal marking. This may allow a medical practitioner to effectively determine whether a medical device is meant to be used for CT injections. The method of the present invention reduces and/or eliminates the chance of over-pressurization of the implanted medical device, i.e., using a low pressure port for a CT injection, which could otherwise cause the medical device to burst or otherwise fail. This helps to prevent unwanted complications or increased procedure time and increases the reliability of identifying the characteristics of the implanted medical device.

As noted above, it is contemplated that the dermal marking 27 of the present invention may be semi-permanent or permanent. A semi-permanent dermal marking 27 is defined as being a dermal marking 27 that can be removed or will degrade, dissolve, or otherwise dissipate at the conclusion of a patient's treatment or after some predetermined period of time. A semi-permanent dermal marking 27 may be applied to the epidermis of the patient's skin 11, which will ensure that the patient is not left with an indefinite marking, because the epidermis is renewable. In this aspect, semi-permanent dermal markings 27 of the present invention are intended to last anywhere from a few days to a few months. Optionally, the dermal marking 27 may be disposable. In another aspect, the dermal marking 27 of the present invention may be applied as one layer or as many layers as desirable on the patient's skin 11.

In one exemplary aspect, the semi-permanent dermal marking 27 can be designed to be visible on the patient's skin 11 for a predetermined period of time, for instance, for such period of time as CT injections are required. After such predetermined period of time, the dermal marking 27 of the present invention can begin to wear off automatically, as the stratum corneum cells of the epidermis flake off. The semi-permanent dermal marking 27 may also be timed to wear off upon the planned or unplanned expiration of the life of the implanted medical device. For example, if an implanted medical device is determined to have a life span of approximately one year, the dermal marking 27 may be designed to last for approximately one year as well, after which it may begin to break down. Such semi-permanent dermal markings 27 may be waterproof, but can be removed with oil-based creams.

Alternatively, the dermal marking 27 of the present invention could be a permanent tattoo that can be used in chronic patients who require long term or indefinite implantation of a medical port, such as throughout the rest of a patient's life. In contrast to a semi-permanent tattoo 27, a permanent tattoo 27 remains upon the patient's skin 11 for an extended period of time, or in perpetuity. In a permanent dermal marking 27, the ink is applied through the epidermis and further into the dermis of the patient's skin 11.

The dermal marking 27 of the present invention may be applied using any medically approved application procedure known in the art. Such methods may include the use of lasers, stencils, automated printing apparatuses such as plotters, ink-jet printers, or tattoo machines, which could be controlled by remote devices or computers. Other methods could include preprinting clear plastic substrates with ink that adheres to the plastic, covering the printing with a backing, and then transferring to the patient's skin, such as decals or labels. Yet other methods well known in the art include iontophoresis, electroporation, chemical permeation enhancement, and combinations thereof, or ultrasound energy, which may be used to transfer pigments from a template of a pattern to be applied to the patient's skin. The dermal marking 27 may also be applied using photo imaging paper and materials, or any other suitable mechanism for applying or transferring a pigmented marking to the surface of the patient's skin. The dermal marking 27 of the present invention is applied externally directly upon the epidermis and/or dermis of the patient's skin 11. The dermal marking 27 of the present invention may be applied before, during, or shortly after implantation of the vascular access port 36.

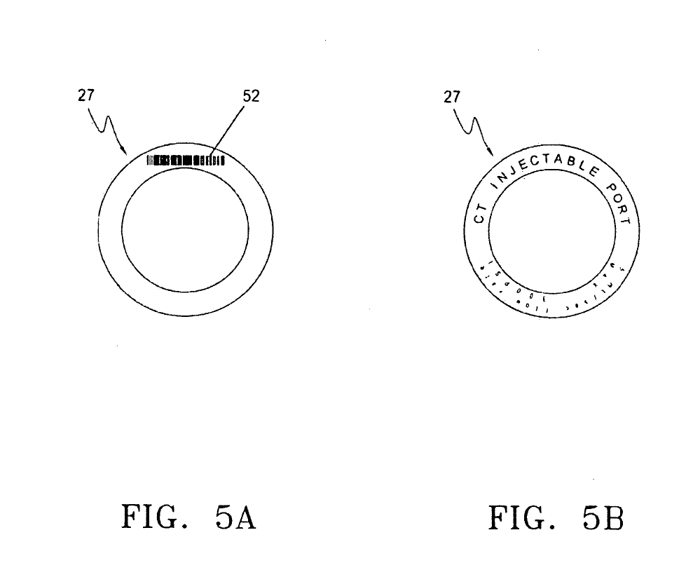

In another aspect of the invention, the method of identifying characteristics of the implanted medical device may comprise scanning a bar code label 52 which can be in machine-readable format, as illustrated in FIG. 5A. In one aspect, the bar code label 52 may contain scannable information pertaining to at least one characteristic of the implanted medical device 36. The bar code label 52 will preferably comply with Uniform Code Council (UCC) quality control barcode standards. Optionally, the bar code label 52 may be the dermal marking 27 itself or may be just a part of the dermal marking 27. This method involves providing an implantable medical device 36 and implanting the medical device 36 beneath a patient's skin 11. The exemplary method further comprises providing at least one dermal marking 27 that is a bar code label 52 or includes a bar code label 52, as described above and applying the dermal marking 27 to the skin 11 of a patient. The applied dermal marking 27 can be scanned on the patient's skin 11 using a scanning device (not shown) and, by perceiving output data produced from the scanning device pertaining to the dermal marking 27, the at least one characteristic of the implanted medical device 36 can be identified.

Alternatively, the method of the present invention may comprise providing an implantable medical device 36, applying the dermal marking 27 to the skin 11 of a patient, implanting the medical device 36 beneath a patient's skin 11, providing at least one dermal marking 27 that is a bar code label 52 or includes a bar code label 52, as described above, scanning the dermal marking 27 on the patient's skin 11 using a scanning device (not shown), perceiving output data from the scanning device pertaining to the dermal marking 27, and identifying at least one characteristic of the implanted medical device 36.

A medical practitioner may use any type of scanning device to scan the bar code label 52 and retrieve scannable information from the bar code label 52, such as, for example and not meant to be limiting, an optical scanner, medical coding machine reader, or the like. In one aspect, the scanning device allows the bar code label 52 to be decoded by using a scanning signal that passes a pulse of light over the bar code label 52 and detects back-scattered light patterns reflected by the bar code label 52. Thus, the coded information is transferred from the printed bar code label 52 to the scanning device. After the data is captured, such as through any number of Auto ID Data Capture (AIDC) systems, the data is converted to useful information that is in readable form, such as for example, on a computer screen or monitor. The computer screen or monitor could be a component of the scanning device itself, or the computer screen or monitor could be separate from the scanning device. The advantage of this method is that the bar code label 52 is capable of conveying more information about the implanted medical device 36 than is possible to convey in the finite surface area of the patient's skin 11 at or in the general vicinity of the implanted medical device 36, using standard numbers, letters, or other symbols.

Although the bar code label 52 is depicted in FIG. 5A as a series of vertical parallel lines, the bar code label 52 may also be composed of any suitable pattern or design. For example, the bar code label 52 may be, but is not limited to, concentric squares or circles or various dot configurations. The inks or pigments used for the bar code label 52 may be any suitable color, such as any of the medically acceptable pigments, inks, or dyes described above. Alternatively, the bar code label 52 could be composed of inks that are only visible using an electronic reader device, such as a bar code reader at certain wavelengths, for instance, at infrared wavelengths or UVA light, also known as black light.

In another aspect of the present invention, a kit can be provided that may comprise, but is not limited to, a container such as a box, tray, bag, pouch, or other similar containers, a sterile, wrapped implantable medical device, such as, but not limited to, a vascular access port assembly 31, and at least one dermal marking 27, as described above. The vascular access port 31 may include at least a port housing 41, a septum 39, a reservoir (not shown), and a catheter 46 that is fluidly connected to the septum 39. The at least one dermal marking 27 may be, for example, a tattoo that is semi-permanent or permanent. A laser device or other conjunctive hardware may be included in the kit or made separately available, in addition to the kit, to be used for the purpose of applying the dermal marking 27 to the patient's skin 11.

The kit may also contain a sealable packaging enclosure that is adapted to receive one or more component parts of the kit and any additional materials necessary for implanting the port, such as a penetrating element, access tube, or the like, or for application of the semi-permanent or permanent dermal marking 27, including instructions for use, and/or additional marketing materials or literature. To this end, instructions optionally included within a kit of the instant invention would, in one aspect, be in substantial accordance with conventional methods of applying medical tattoos, as well as conventional methods of implanting vascular access ports, known to one of ordinary skill in the art.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein. This completes the description of the preferred and alternate embodiments of the invention.

What is claimed is:

1. A method of identifying at least one characteristic of an implantable medical device, comprising:
a) providing at least one implantable medical device;
b) implanting the medical device in a patient;
c) providing at least one dermal marking, wherein the dermal marking comprises information disclosing at least one characteristic of the medical device;
d) applying the at least one dermal marking to the patient's skin;
e) perceiving the dermal marking on the patient's skin; and
f) identifying the at least one characteristic of the implanted medical device based on the dermal marking.

2. The method of claim 1, wherein the method further comprises applying the at least one dermal marking to the patient's skin at or near the vicinity of the implanted medical device.

3. The method of claim 1, wherein the implantable medical device is a vascular access port.

4. The method of claim 1, wherein the dermal marking is semi-permanent.

5. The method of claim 1, wherein the dermal marking is permanent.

6. The method of claim 1, wherein the dermal marking is in the form of a tattoo.

7. The method of claim 1, wherein the at least one characteristic is CT injection capability.

8. The method of claim 1, wherein the at least one characteristic is the device type.

9. A method of identifying at least one characteristic of an implantable medical device, comprising:
a) providing at least one implantable medical device;
b) implanting the medical device in a patient;
c) providing at least one dermal marking, wherein the dermal marking comprises scannable information disclosing at least one characteristic of the medical device;
d) applying the at least one dermal marking to the patient's skin;
e) providing a scanning device;
f) scanning the dermal marking on the patient's skin using the scanning device;
g) perceiving output data produced from the scanning device pertaining to the dermal marking; and
h) identifying the at least one characteristic of the implanted medical device based on the dermal marking.

10. The method of claim 9, wherein the method further comprises applying the at least one dermal marking to the patient's skin at or near the vicinity of the implanted medical device.

11. The method of claim 9, wherein the implantable medical device is a vascular access port.

12. The method of claim 9, wherein the dermal marking is semi-permanent.

13. The method of claim 9, wherein the dermal marking is permanent.

14. The method of claim 9, wherein the dermal marking is in the form of a tattoo.

15. The method of claim 9, wherein the at least one characteristic is CT injection capability.

16. The method of claim 9, wherein the at least one characteristic is the device type.

17. A method of identifying the characteristics of an implantable medical device, comprising:
a) providing at least one implantable medical device;
b) implanting the medical device in a patient;
c) providing at least one dermal marking, wherein the dermal marking comprises information disclosing at least one characteristic of the medical device, and wherein the at least one characteristic comprises CT injection capability;
d) applying the at least one dermal marking to the patient's skin;
e) perceiving the dermal marking on the patient's skin; and
f) identifying the at least one characteristic of the implanted medical device based on the dermal marking.

18. The method of claim 17, further comprising:
e) providing a scanning device;
f) scanning the dermal marking on the patient's skin using the scanning device;
f) perceiving output data produced from the scanning device pertaining to the dermal marking; and
g) identifying the at least one characteristic of the implanted medical device based on the dermal marking.

19. A dermal marking for use with an implantable medical device, wherein the dermal marking comprises information disclosing at least one characteristic of the medical device, and wherein the at least one characteristic comprises CT injection capability.

20. The dermal marking of claim 19, wherein the implantable medical device is a vascular access port.

21. The dermal marking of claim 19, wherein the dermal marking is semi-permanent.

22. The dermal marking of claim 19, wherein the dermal marking is permanent.

23. The dermal marking of claim 19, wherein the dermal marking is in the form of a tattoo.

24. A kit comprising:
(a) a container;
(b) at least one insertable or implantable medical device;
(c) at least one dermal marking for use with the medical device;
(d) instructions for use; and
(e) a sealable packaging enclosure.

25. The kit of claim 24, further comprising a catheter connected or connectable to the implantable medical device.
